(19) United States
(12) Patent Application Publication
(10) Pub. No.: US 2013/0138081 A1
(43) Pub. Date: May 30, 2013

(54) METHOD AND MEDICAL DEVICE HAVING TISSUE ENGAGING MEMBER FOR DELIVERY OF A THERAPEUTIC AGENT

(71) Applicant: ABBOTT CARDIOVASCULAR SYSTEMS INC., Santa Clara, CA (US)

(72) Inventors: John J. Stankus, Campbell, CA (US); Michael J. Leonard, Palo Alto, CA (US); Stephen D. Pacetti, San Jose, CA (US)

(73) Assignee: ABBOTT CARDIOVASCULAR SYSTEMS INC., Santa Clara, CA (US)

(21) Appl. No.: 13/741,049

(22) Filed: Jan. 14, 2013

Related U.S. Application Data
(63) Continuation of application No. PCT/US2011/043812, filed on Jul. 13, 2011.
(60) Provisional application No. 61/365,197, filed on Jul. 16, 2010.

(51) Int. Cl. A61M 25/10

(52) U.S. Cl. CPC A61M 25/10
USPC 604/509; 604/104

(57) ABSTRACT

Method of delivering a therapeutic agent includes delivering at least a portion of a medical device within a vasculature. The medical device includes a tubular member having a proximal end and distal end defining a longitudinal axis therebetween, an expandable member proximate the distal end of the tubular member, a tissue engaging member proximate the expandable member, a sheath disposed over the tissue engaging member, and a therapeutic agent disposed on at least the expandable member or the tissue engaging member. The method further includes deploying the tissue engaging member at a select location by displacement of the sheath relative the tissue engaging member, inflating the expandable member to engage the therapeutic agent with a vessel wall, deflating the expandable member, and withdrawing the medical device from the vasculature.

### CROSS-REFERENCE TO RELATED PRIORITY

This application is a continuation of International Patent Application Serial No. PCT/US2011/043812 filed Jul. 13, 2011 and claims priority to U.S. Provisional Patent Application Ser. No. 61/365,197 entitled “Method and Medical Device Having Tissue Engaging Member for Delivery of a Therapeutic Agent” filed on Jul. 16, 2010, which are hereby incorporated by reference in their entireties herein.

### BACKGROUND OF THE DISCLOSED SUBJECT MATTER

1. Field of the Disclosed Subject Matter

The disclosed subject matter is related to the delivery of therapeutic agents from an interventional medical device. More particularly, the presently disclosed subject matter relates to an interventional device including an expandable member, such as a balloon, and a tissue engaging member for the delivery of a therapeutic agent to a vasculature.

2. Description of Related Subject Matter

Atherosclerosis is a syndrome affecting arterial blood vessels. It is characterized by a chronic inflammatory response in the walls of arteries, which is in large part due to the accumulation of lipid, macrophages, foam cells and the formation of plaque in the arterial wall. Atherosclerosis is commonly referred to as hardening of the arteries, although the pathophysiology of the disease manifests itself with several different types lesions ranging from fibrotic to lipid laden to calcific. Angioplasty is a vascular interventional technique involving mechanically widening an obstructed blood vessel, typically caused by atherosclerosis.

During angioplasty, a catheter having a folded balloon is inserted into the vasculature of the patient and is passed to the narrowed location of the blood vessel at which point the balloon is inflated to the desired size by fluid pressure. Percutaneous coronary intervention (PCI), commonly known as coronary angioplasty, is a therapeutic procedure to treat the stenotic regions in the coronary arteries of the heart, often found in coronary heart disease. In contrast, peripheral angioplasty, commonly known as percutaneous transluminal angioplasty (PTA), generally refers to the use of mechanical widening of blood vessels other than the coronary arteries. PTA is most commonly used to treat narrowing of the leg arteries, especially, the iliac, external iliac, superficial femoral and popliteal arteries. PTA can also treat narrowing of carotid and renal arteries, veins, and other blood vessels.

Although the blood vessel is often successfully widened by angioplasty, sometimes the treated region of the blood vessel undergoes vasospasm, or abrupt closure after balloon inflation or dilatation, causing the blood vessel to collapse after the balloon is deflated or shortly thereafter. One solution to such collapse is stenting the blood vessel to prevent collapse. Dissection, or perforation, of the blood vessel is another complication of angioplasty that can be improved by stenting. A stent is a device, typically a metal tube or scaffold that is inserted into the blood vessel after, or concurrently with angioplasty, to hold the blood vessel open.

While the advent of stents eliminated many of the complications of abrupt vessel closure after angioplasty procedures, within about six months of stenting a re-narrowing of the blood vessel can form, a condition known as restenosis. Restenosis was discovered to be a response to the injury of the angioplasty procedure and is characterized by a growth of smooth muscle cells and extracellular matrix—analogous to a scar forming over an injury. To address this condition, drug eluting stents were developed to reduce the reoccurrence of blood vessel narrowing after stent implantation. A drug eluting stent is a stent that has been coated with a drug, often in a polymeric carrier, that is known to interfere with the process of re-narrowing of the blood vessel (restenosis). Examples of various known drug eluting stents are disclosed in U.S. Pat. Nos. 5,649,977; 5,464,650; 5,591,227; 7,378,105; 7,445,792; and 7,335,227, each of which are hereby incorporated by reference in their entirety. However, a drawback of drug eluting stents is a condition known as late stent thrombosis. This is an event where a blood clot forms inside the stent, which can occlude blood flow.

Drug coated balloons are believed to be a viable alternative to drug eluting stents in the treatment of atherosclerotic lesions. In a study which evaluated restenosis, and the rate of major adverse cardiac events such as heart attack, bypass, repeat stenosis, or death in patients treated with drug coated balloons and drug eluting stents, the patients treated with drug coated balloons experienced only 3.7 percent restenosis and 4.8 percent MACE (material adverse coronary events) as compared to patients treated with drug eluting stents, in which restenosis was 20.8 percent and 22.0 percent MACE rate. (See, PEPCAD II study, Rotenburg, Germany).

However, drug coated balloons present certain unique challenges. For example, the drug carried by the balloon needs to remain on the balloon during delivery to the lesion site, and release from the balloon surface to the blood vessel wall when the balloon is expanded inside the blood vessel. For coronary procedures, the balloon is typically inflated for less than one minute, typically about thirty seconds. The balloon inflation time may be longer for a peripheral procedure, however typically even for peripheral procedures the balloon is expanded for less than 5 minutes. Due to the short duration of contact between the drug coated balloon surface and the blood vessel wall, the balloon coating must exhibit efficient therapeutic agent transfer and/or efficient drug release during inflation. Thus, there are challenges specific to drug delivery via a drug coated or drug eluting balloon that are not present with a drug eluting stent.

Thus there remains a need, and an aim of the disclosed subject matter is directed towards, a medical device and method for increasing the delivery of a therapeutic agent to a vasculature. Furthermore, there remains a need for a more controlled angioplasty procedure.

### SUMMARY OF THE DISCLOSED SUBJECT MATTER

The purpose and advantages of the disclosed subject matter will be set forth in and are apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a method of delivering a therapeutic agent. The method includes delivering at least a portion of a medical device within a vasculature. The medical device includes a tubular member having a proximal end and distal end defining a longitudinal axis therebetween, an expandable member proximate the distal end of the tubular member, a tissue engaging member proximate the expandable member, a sheath disposed over the tissue engaging member, and a therapeutic agent disposed on at least the expandable member or the tissue engaging member. The method further includes deploying the tissue engaging member at a select location by displacement of the sheath relative the tissue engaging member, inflating the expandable member to engage the therapeutic agent with a vessel wall, deflating the expandable member, and withdrawing the medical device from the vasculature.

The disclosed subject matter also includes a medical device. The medical device includes a tubular member having a proximal end and distal end defining a longitudinal axis therebetween, an expandable member proximate the distal end of the tubular member, a tissue engaging member proximate the expandable member, a retractable sheath disposed over the tissue engaging member, and a therapeutic agent disposed on at least the expandable member or the tissue engaging member. The tissue engaging member is configured for expansion at a select location. The method and medical device can include a number of the features described in greater detail below.

Further in accordance with the disclosed subject matter, an alternative method of delivering a therapeutic agent is provided. The method includes delivering at least a portion of a medical device within a vasculature. The medical device includes a tubular member having a proximal end and distal end defining a longitudinal axis therebetween, an expandable member proximate the distal end of the tubular member, a tissue engaging member proximate the expandable member and having at least one wire disposed within at least one lumen extending along the longitudinal axis of the tubular member, and a therapeutic agent disposed on at least the expandable member or the tissue engaging member. The method further includes deploying the at least one wire of the tissue engaging member from within the at least one lumen at a select location, inflating the expandable member to engage the therapeutic agent with a vessel wall, deflating the expandable member, and withdrawing the medical device from the vasculature. The method can include any of the features described in greater detail below.

The disclosed subject matter also includes a medical device. The medical device includes a tubular member having a proximal end and distal end defining a longitudinal axis therebetween, an expandable member proximate the distal end of the tubular member, at least one lumen extending along the longitudinal axis of the tubular member, a tissue engaging member having at least one wire slidingly disposed within the at least one lumen for deployment and retraction of the at least one tissue engaging member, and a therapeutic agent disposed on at least the expandable member or the tissue engaging member. The tissue engaging member is configured for expansion at a select location. The medical device can have any of the features described in greater detail below.

The disclosed subject matter also includes a method of treating a vasculature. The method includes delivering at least a portion of a medical device within a vasculature. The medical device includes a tubular member having a proximal end and distal end defining a longitudinal axis therebetween, an expandable member proximate the distal end of the tubular member, a tissue engaging member proximate the expandable member, and a sheath disposed over the tissue engaging member. The method further includes deploying the tissue engaging member at a select location by displacement of the sheath relative the tissue engaging member, inflating the expandable member to engage the expandable member with a vessel wall, deflating the expandable member, and withdrawing the medical device from the vasculature.

The disclosed subject matter also includes a medical device. The medical device includes a tubular member having a proximal end and distal end defining a longitudinal axis therebetween, an expandable member proximate the distal end of the tubular member, a tissue engaging member proximate the expandable member, and a retractable sheath disposed over the tissue engaging member. The tissue engaging member is configured for expansion at a select location. The method and medical device can include a number of the features described in greater detail below.

Further in accordance with the disclosed subject matter, an alternative method of treating a vasculature is provided. The method includes delivering at least a portion of a medical device within a vasculature. The medical device includes a tubular member having a proximal end and distal end defining a longitudinal axis therebetween, an expandable member proximate the distal end of the tubular member, and a tissue engaging member proximate the expandable member and having at least one wire disposed within at least one lumen extending along the longitudinal axis of the tubular member. The method further includes deploying the at least one wire of the tissue engaging member from within the at least one lumen at a select location, inflating the expandable member to engage the expandable member with a vessel wall, deflating the expandable member, and withdrawing the medical device from the vasculature. The method can include any of the features described in greater detail below.

The disclosed subject matter also includes a medical device. The medical device includes a tubular member having a proximal end and distal end defining a longitudinal axis therebetween, an expandable member proximate the distal end of the tubular member, at least one lumen extending along the longitudinal axis of the tubular member, and a tissue engaging member having at least one wire slidingly disposed within the at least one lumen for deployment and retraction of the at least one tissue engaging member. The tissue engaging member is configured for expansion at a select location. The medical device can have any of the features described in greater detail below.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

FIG. 1 is a schematic side view with partial cross-section of a representative balloon catheter in accordance with the disclosed subject matter.

FIG. 1A is a cross-sectional view taken along lines A-A in FIG. 1.

FIG. 1B is a cross-sectional view taken along lines B-B in FIG. 1.

FIG. 2 is a schematic side view of an expandable member and tissue engaging member in accordance with the disclosed subject matter.

FIG. 3 is a schematic side view of another expandable member and tissue engaging member in accordance with the disclosed subject matter.

FIG. 4 is a schematic side view of another expandable member and tissue engaging member in accordance with the disclosed subject matter.

FIG. 5 is schematic side view of another expandable member and tissue engaging member in accordance with the disclosed subject matter.

FIGS. 6A and 6B are a schematic side view of an expandable member, a tissue engaging member, and a sheath in accordance with the disclosed subject matter.

FIG. 7 is a schematic side view of the expandable member, tissue engaging member, and sheath of FIG. 6B showing the sheath being moved relative to the expandable member into position over the tissue engaging member.

FIG. 8 is a schematic side view of an expandable member and a tissue engaging member extending from a lumen extending along a tubular member in accordance with the disclosed subject matter.

FIG. 9 is a schematic side view of another expandable member and tissue engaging member extending from a lumen extending along a tubular member in accordance with the disclosed subject matter.

### DETAILED DESCRIPTION OF THE PREFERRED EMBODIMENT

Reference will now be made in detail to the preferred embodiments of the disclosed subject matter, an example of which is illustrated in the accompanying drawing. The method and corresponding steps of the disclosed subject matter will be described in conjunction with the detailed description of the system.

As disclosed herein, the devices and methods presented can be used for treating the lumen of a patient. In particular, the disclosed subject matter is particularly suited for treatment of the cardiovascular system of a patient, such as performance of angioplasty and delivery of a therapeutic agent to a vasculature.

In accordance with the disclosed subject matter, a method of delivering a therapeutic agent comprises delivering at least a portion of a medical device within a vasculature. The medical device includes a tubular member having a proximal end and distal end defining a longitudinal axis therebetween, an expandable member proximate the distal end of the tubular member, a tissue engaging member proximate the expandable member, a sheath disposed over the tissue engaging member, and a therapeutic agent disposed on at least the expandable member or the tissue engaging member. The method further includes deploying the tissue engaging member at a select location by displacement of the sheath relative the tissue engaging member, inflating the expandable member to engage the therapeutic agent with a vessel wall, deflating the expandable member, and withdrawing the medical device from the vasculature.

The disclosed subject matter also includes a medical device. The medical device includes a tubular member having a proximal end and distal end defining a longitudinal axis therebetween, an expandable member proximate the distal end of the tubular member, a tissue engaging member proximate the expandable member, a retractable sheath disposed over the tissue engaging member, and a therapeutic agent disposed on at least the expandable member or the tissue engaging member. The tissue engaging member is configured for expansion at a select location. The medical device will be described in conjunction with the method for purpose of understanding.

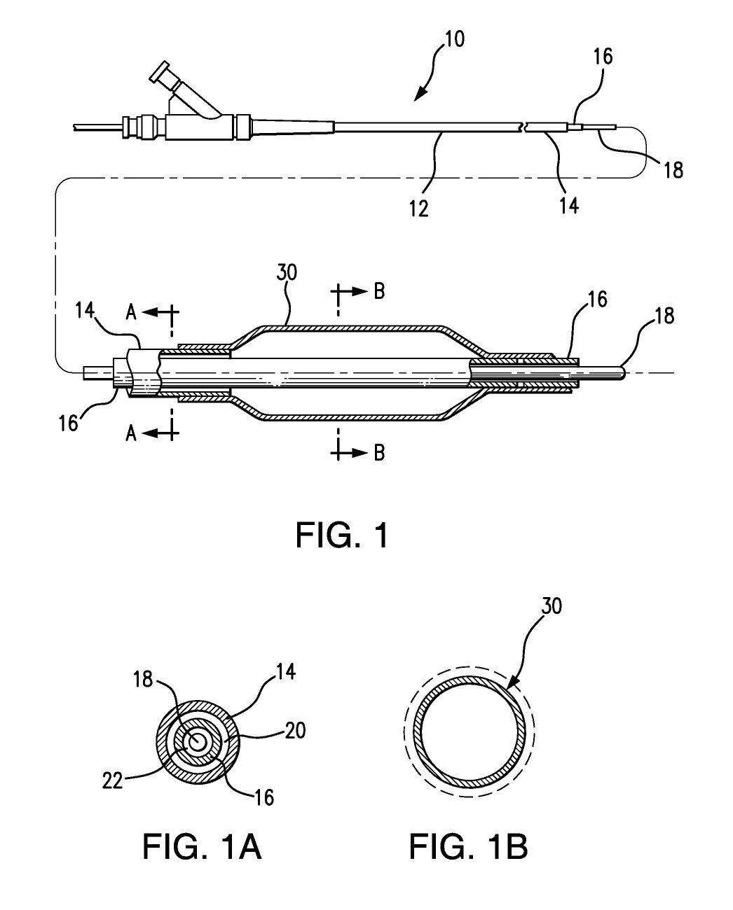

For purpose of explanation and illustration, and not limitation, an exemplary embodiment of a medical device, at least a portion of which is delivered within a vasculature, is shown schematically in FIG. 1. Particularly, and as illustrated, the medical device embodied herein can be a balloon catheter 10, which includes an tubular member or elongated catheter shaft 12 having a proximal end and distal end defining a longitudinal axis therebetween and an expandable member 30 located proximate the distal end of the catheter shaft. The expandable member, or balloon as depicted herein, has an outer surface and an inner surface disposed at the distal end portion of the catheter shaft.

The elongated catheter shaft 12 comprises an outer tubular member 14 and an inner tubular member 16. The outer tubular member 14 defines an inflation lumen 20 disposed between the proximal end portion and the distal end portion of the catheter shaft 12. Specifically, as illustrated in FIG. 1A, the coaxial relationship of this representative embodiment defines an annular inflation lumen 20 between the inner tubular member 16 and the outer tubular member 14. The expandable member 30 is in fluid communication with the inflation lumen 20. The inflation lumen can supply an inflation medium under positive pressure and can withdraw the inflation medium, i.e. provide negative pressure, from the expandable member. The expandable member 30 can thus be inflated and deflated. The elongated catheter is sized and configured for delivery within a vasculature and particularly through a tortuous anatomy, and can further include a guidewire lumen 22 that permits it to be delivered over a guidewire 18. As illustrated in FIG. 1A, the inner tubular member 16 defines the guidewire lumen 22 for the guidewire 18. Although FIGS. 1 and 1b illustrate the guidewire lumen as having an over-the-wire (OTW) construction, the guidewire lumen can be configured as a rapid-exchange (RX) construction, as is well known in the art.

A wide variety of expandable members 30, such as balloons, and constructs are known and suitable for use in accordance with the disclosed subject matter. For example, the expandable member can be made from polymeric material such as compliant, non-compliant or semi-compliant polymeric material or polymeric blends.

In one embodiment, the polymeric material is compliant such as, but not limited to, a polyamide/polyether block copolymer (commonly referred to as PEBA or polyether-block-amide). Preferably, the polyamide and polyether segments of the block copolymers can be linked through amide or ester linkages. The polyamide block can be selected from various aliphatic or aromatic polyamides known in the art. Preferably, the polyamide is aliphatic. Some non-limiting examples include nylon 12, nylon 11, nylon 9, nylon 6, nylon 6/12, nylon 6/11, nylon 6/9, and nylon 6/6. Preferably, the polyamide is nylon 12. The polyether block can be selected from various polyethers known in the art. Some non-limiting examples of polyether segments include poly(tetramethylene ether), tetramethylene ether, polyethylene glycol, polypropylene glycol, poly(pentamethylene ether) and poly(hexamethylene ether). Commercially available PEBA material can also be utilized such as for example, PEBAX® materials supplied by Arkema (France). Various techniques for forming a balloon from polyamide/polyether block copolymer are known in the art. One such example is disclosed in U.S. Pat. No. 6,406,457 to Wang, the disclosure of which is incorporated by reference in its entirety.

In another embodiment, the balloon material is formed from polyamides. Preferably, the polyamide has substantial tensile strength, is resistant to pin-holing even after folding and unfolding, and is generally scratch resistant, such as those disclosed in U.S. Pat. No. 6,500,148 to Pinchuk, the disclosure of which is incorporated herein by reference in its entirety. Some non-limiting examples of polyamide materials suitable for the balloon include nylon 12, nylon 11, nylon 9, nylon 69 and nylon 66. Preferably, the polyamide is nylon 12. Other suitable materials for constructing non-compliant balloons are polyesters such as polyethylene terephthalate) (PET), Hytrel thermoplastic polyester, and poly(ethylene.

In another embodiment, the balloon is formed of a polyurethane material, such as TECOTHANE® (Thermedics). TECOTHANE® is a thermoplastic, aromatic, polyether polyurethane synthesized from methylene disocyanate (MDI), polytetramethylene ether glycol (PTMEG) and 1,4 butanediol chain extender. TECOTHANE® grade 1065D is presently preferred, and has a Shore durometer of 65D, an elongation at break of about 300%, and a high tensile strength at yield of about 10,000 psi. However, other suitable grades can be used, including TECOTHANE® 1075D, having a Shore D hardness of 75. Other suitable compliant polymeric materials include ENGAGE® {DuPont Dow Elastomers (an ethylene alpha-olefin polymer)) and EXACT® (Exxon Chemical), both of which are thermoplastic polymers. Other suitable compliant materials include, but are not limited to, elastomeric silicones, latexes, and urethanes.

The compliant material can be cross linked or uncrosslinked, depending upon the balloon material and characteristics required for a particular application. The presently preferred polyurethane balloon materials are not crosslinked. However, other suitable materials, such as the polyolefinic polymers ENGAGE® and EXACT®, are preferably crosslinked. By crosslinking the balloon compliant material, the final inflated balloon size can be controlled. Conventional crosslinking techniques can be used including thermal treatment and E-beam exposure. After crosslinking, initial pressurization, expansion, and preshrinking, the balloon will thereafter expand in a controlled manner to a reproducible diameter in response to a given inflation pressure, and thereby avoid overexpanding the balloon to an undesirably large diameter.

In one embodiment, the balloon is formed from a low tensile set polymer such as a silicone-polyurethane copolymer. Preferably, the silicone-polyurethane is an ether urethane and more specifically an aliphatic ether urethane such as PURSIL AL 575A and PURSIL AL10, (Polymer Technology Group), and ELAST-EON 3-70A (Elastomedics), which are silicone polyether urethane copolymers, and more specifically, aliphatic ether urethane cosiloxanes. In an alternative embodiment, the low tensile set polymer is a diene polymer. A variety of suitable diene polymers can be used such as, but not limited to, an isoprene such as an AB and ABA poly(styrene-block-isoprene), a neoprene, an AB and ABA poly(styrene-block-butadiene) such as styrene butadiene styrene (SBS) and styrene butadiene rubber (SBR), and 1,4-polybutadiene. Preferably, the diene polymer is an isoprene including isoprene copolymers and isoprene block copolymers such as poly(styrene-block-isoprene). A presently preferred isoprene is a styrene-isoprene-styrene block copolymer, such as Kraton 1161K available from Kraton, Inc. However, a variety of suitable isoprenes can be used including HT 200 available from Apex Medical, Kraton R 310 available from Kraton, and isoprene (i.e., 2-methyl-1,3-butadiene) available from Dupont Elastomers. Neoprene grades useful in the disclosed subject matter include HT 501 available from Apex Medical, and neoprene (i.e., polychloroprene) available from Dupont Elastomers, including Neoprene G, W, T and A types available from Dupont Elastomers. Examples of other balloon and catheter embodiments which can be employed in accordance with the disclosed subject matter include U.S. Pat. Nos. 4,748,982; 5,496,346; 5,626,600; 5,300,085; and 6,406,457 and application Ser. Nos. 12/371,426; 11/539,944; and 12/371,422, each of which is hereby incorporated by reference in its entirety.

In accordance with another aspect of the disclosed subject matter, the outer surface of the balloon can be modified. In this regard, the balloon surface can include a textured surface, roughened surface, voids, spines, channels, dimples, pores, or microcapsules or a combination thereof.

In accordance with the disclosed subject matter, the expandable member of the medical device can have a plurality of folds defined therein. For example, a number of conventional balloon catheters include such folds, so as to have a folded configuration and a fully expanded configuration. Generally, the formation of folds can be performed using heat and pressure to form or define creases in the material of the balloon. Examples of such folded balloons are disclosed, for purpose of illustration in U.S. Pat. Nos. 6,494,906; 6,478,807; and 5,911,452, each of which is hereby incorporated by reference in its entirety.

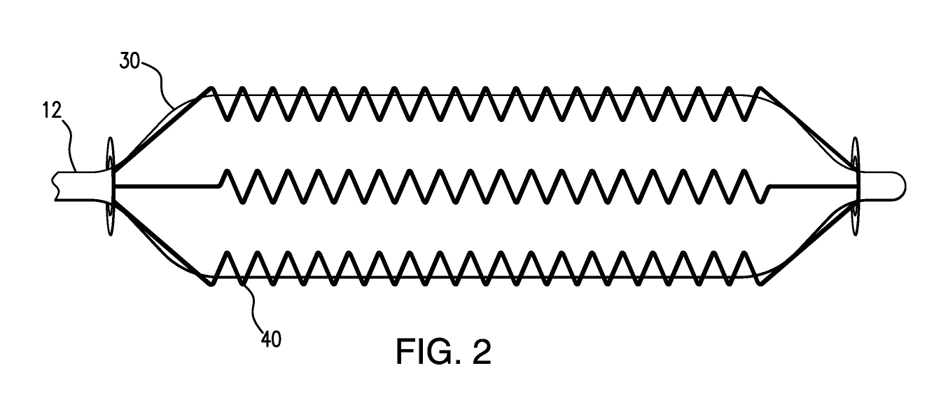

For purpose of explanation and illustration, and not limitation, an exemplary embodiment of the expandable member and a tissue engaging member is shown schematically in FIG. 2. The tissue engaging member 40 is locate proximate the expandable member 30, and preferably, as shown is FIG. 2, is disposed over the expandable member 30. The tissue engaging member is in a collapsed configuration for delivery and an expanded configuration for engagement with the vessel wall. The issue engaging member can be a self-expanding structure. In that regard, a retractable sheath can be provided to operatively engage a tissue engaging member for conversion between expanded and unexpanded configurations upon displacement of the retractable sheath, as will be described in more detail below. Alternatively, the tissue engaging member can be shape-set or thermally trained to be in the collapsed state, such that it is expanded by inflation of the expandable member. In such an embodiment, upon deflation, the tissue engaging member will return to the smaller collapsed profile.

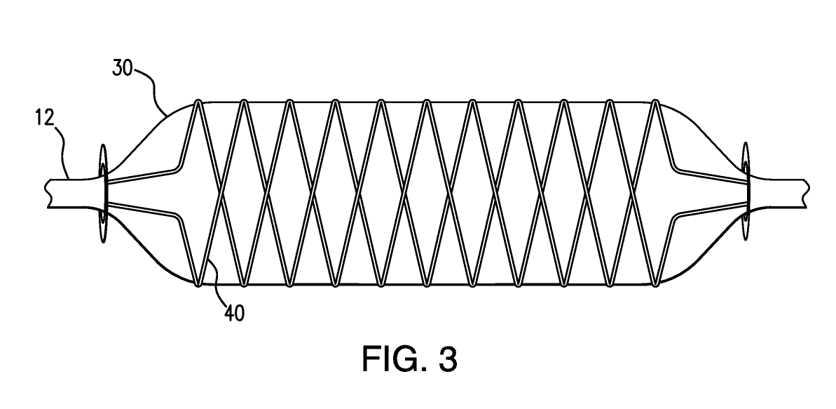

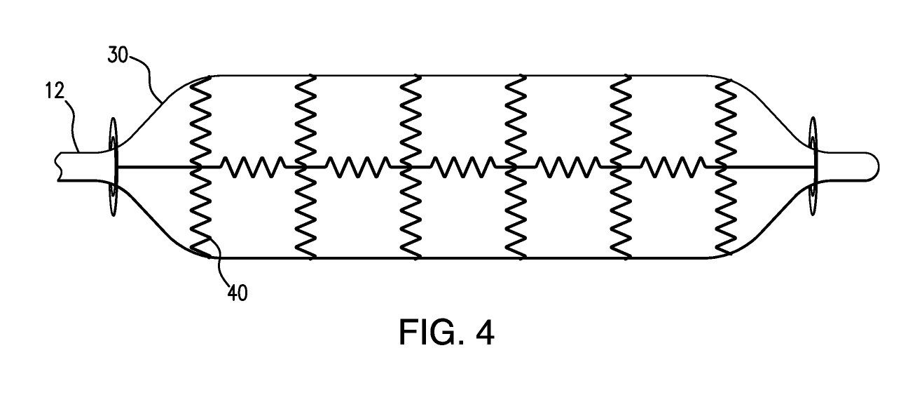

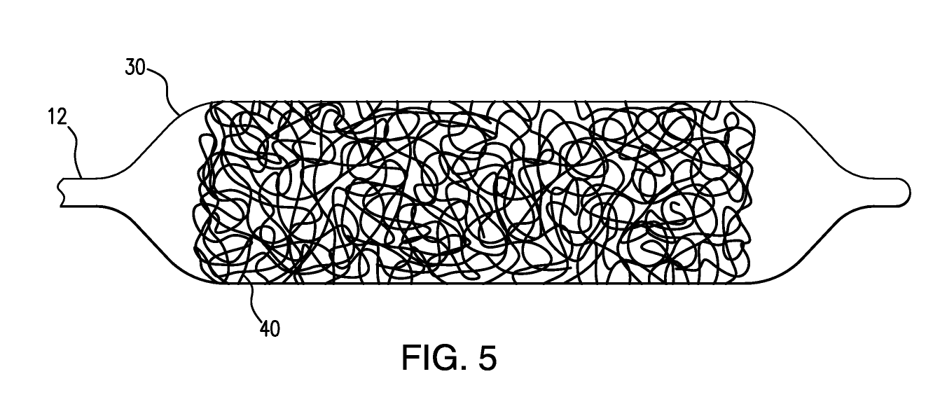

The tissue engaging member can have any of a number of suitable configurations. For example and not limitation, the tissue engaging member can include a plurality of wire elements extending along a length of the expandable member. Depending upon desired performance characteristics, the elements or wires can be straight {i.e., linear) or non-liner (e.g., wavy). Also, the plurality of wires can be independent or can be interconnected. As embodied herein, the tissue engaging member includes a plurality of continuous longitudinal wires that can assist in the transfer of drug to the vessel wall, as well as collapsing of the expandable member when desired. The tissue engaging member can be a zig-zag design to increase drug uptake into the vessel wall. For the purpose of illustration and not limitation, reference is made to FIGS. 2 through 5 to show alternate configurations of the tissue engaging members. As shown in FIG. 2, the tissue engaging member 40 can be a set of non-linear wires extending substantially over the entire length of the expandable member 30. Alternatively, the tissue engaging member 40 can be a set of wires that interconnect in a pattern, such as a braid, shown in FIG. 3, for the purpose of illustration and not limitation. As shown in FIG. 4, the tissue engaging member 40 can be a set of elements interconnected by circumferential rings made of nonlinear wires. As shown in FIG. 5, the tissue engaging member can be a woven or electrospun pattern. Other configurations are possible such as any number of configurations or designs that are well known in the art for stents. If interconnected, the wires of the tissue engaging member can be joined by a variety of conventional means, such as for example and without limitation, epoxy, fusion, welded, riveted, hoops and knots, or formed of one piece. If the expandable member of the medical device has a plurality of folds defined therein, the wires of the tissue engaging member can be located within the folds of the expandable member, as described in detail in concurrently filed U.S. Provisional Application No. 61/365,203 entitled “Medical Device Having Tissue Engaging Member and Method for Delivery of a Therapeutic Agent” which is hereby incorporated by reference in its entirety.

In accordance with another aspect of the disclosed subject matter, the tissue engaging member has a tissue engaging member to artery ratio, which represents the percent of the luminal area occupied by the tissue engaging member when expanded against the luminal vessel wall, between about 1 to about 50% and preferably between about 2.5 to about 25%.

In accordance with another aspect of the disclosed subject matter, the tissue engaging member can be fixed at least one of either the proximal or distal end. For example, the proximal end of the tissue engaging member can be fixedly attached to the tubular member proximate a proximal end of the expanded member. Additionally or alternatively, the distal end of the tissue engaging member can be fixedly attached to the tubular member proximate a distal end of the expanded member. If fixed only at the distal end, care should be taken to prevent the sheath from retracting too far such that the proximal end of the tissue engaging member is no longer held in place by the sheath, because it could be difficult to reengage the sheath over the tissue engaging member. The tissue engaging member, or each individual element or wire, can be joined to the tubular member by any known means, such as adhesively bonded, thermo bonded, welded, crimped, etc. For example, the tissue engaging member can include a collar encircling and/or joined to the tubular member. Additionally or alternatively, the wires of the tissue engaging member can be slipped or mounted into small formations on the tubular member. Furthermore, the tissue engaging member can be coupled to the expandable member such that the tissue engaging member collapses upon deflation of the expandable member. The tissue engaging member can be coupled to the balloon using a variety of known techniques such as, but not limited to, using solvents or adhesives, or by formations provided on the surface of the expandable member to capture or engage the wires or elements.

The tissue engaging member can be made of a variety of suitable materials. For example, the tissue engaging member can be metallic, a polymer, an elastomer, or a metallic alloy. Non-limiting examples of suitable materials include nitinol, elgiloy, stainless steel, cobalt-chromium, alloys thereof, and combinations thereof. In the case of cobalt-chromium alloys and stainless steel alloys, it is preferred to work harden the materials to provide the desired elasticity for expansion. Suitable polymers include polyethylene, polypropylene, poly(ethylene terephthalate), Dytrel, polyurethane, nylon-6, nylon-66, nylon-12, PEBAX, poly(vinylidene fluoride), poly(tetrafluoroethylene), or poly(vinylidene fluoride-co-hexafluoropropylene). If a metallic or polymer or other suitable material is used, the tissue engaging member can be laser cut from a single tube. In one embodiment, the tissue engaging member can be laser cut at the fully expanded size and then fused to the expandable member, for example by placing the tissue engaging member in a constraining tube, inflating the expandable member inside the balloon, and then heating the tube to fuse the tissue engaging member to the expandable member.

Each element or wire of the tissue engaging member can have any suitable dimensions, for example from about 0.05 microns to about 250 microns in diameter, width, and/or height. The elements or wires of the tissue engaging member can have a cross sectional configuration of a variety of shapes and ratios of width to height depending upon desired performance characteristics. Non-limiting examples of suitable cross section configurations include circular, triangular, rectangular, square, or other polygonal cross section configurations. Using a tissue engaging member in accordance with the disclosed subject matter allows for more design freedom and the ability to make wires of an optimal configuration when compared to a conventional stents because the expandable member does not remain in the body after the medical procedure is complete.

The tissue engaging member can include a coating disposed on the outer surface thereof. The coating can include a therapeutic agent, among other components, as described below or more detail.

In accordance with another aspect of the disclosed subject matter, the tissue engaging member can include protrusions or other raised surfaces configured to contact or penetrate the arterial wall of a vessel, which can increase the uptake of the therapeutic agent and provide a more uniform injury to the vessel wall. A coating containing therapeutic agent, and/or other components as described in more detail below, can be disposed on the protrusions such that when expanded, the coating and/or therapeutic agent coats the tissue of the arterial wall. Additionally or alternatively, the surface of the tissue engaging member can be roughened to provide better penetration into the wall of the vessel to enhance drug transfer.

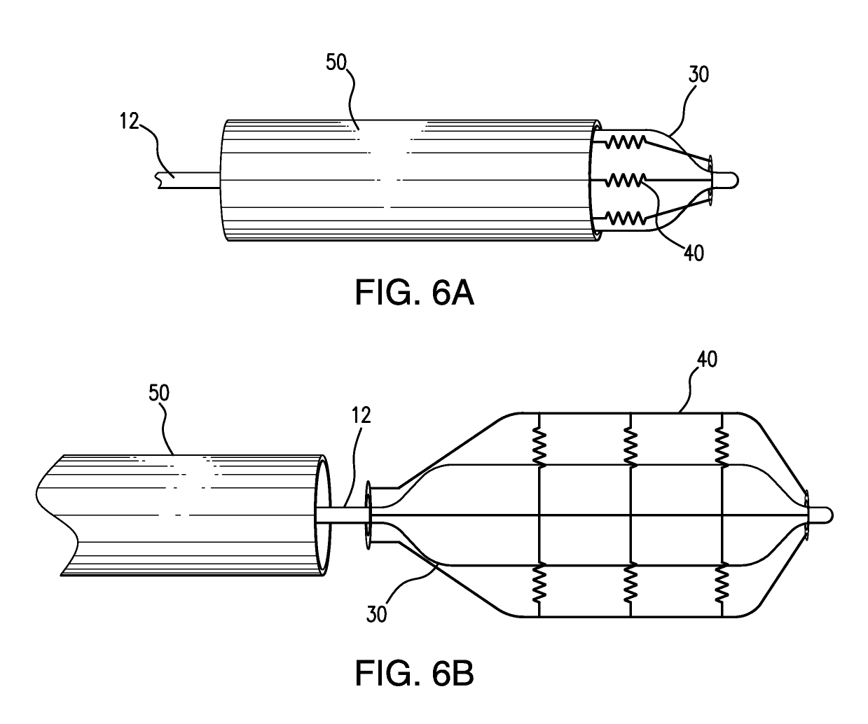

As previously noted, and in accordance with another aspect of the disclosed subject matter, a sheath can be provided. For example, the sheath can prevent the release of drug from the expandable member prior to deployment at the desired site. Additionally, if the tissue engaging member is self-expanding, the sheath can facilitate maintaining the tissue engagement member in the collapsed state prior to deployment at the desired site. For purpose of explanation and illustration, and not limitation, an exemplary embodiment including an expandable member, a self-expanding tissue engaging member, and a sheath is shown schematically in FIGS. 6a and 6b. The sheath 50 is initially disposed over the tissue engaging member 40, as shown in FIG. 6a. The sheath 50 protects the tissue engaging member and the coating of therapeutic agent during delivery of the expandable member through the body lumen to the target site, such that drug loss and injury to the vessel is minimized. The sheath can be utilized to protect the coating containing therapeutic agent from releasing from the expandable member during the movement of the medical device through the body lumen. Furthermore, the sheath can also protect the coating during shipping and storage before use.

Preferably, the sheath is formed from one or more layer of polymeric material known in the art. Preferably the sheath is formed of high density polyethylene (HDPE), fluorinated ethylene propylene (FEP), polytetrafluoroethylene (PTFE), nylons, polyether block amide (PEBAX), polyurethane, polyester, polypropylene, or blends or composites thereof. The sheath can have any configuration such that the sheath is disposed over the tissue engaging member. For example, the sheath preferably is a cylindrical tube extending over substantially the entire length of the tissue engaging member.

In another embodiment, an elastic sheath that conforms to the balloon upon expansion can be used. Such elastic sheaths can be porous or include apertures along a portion thereof. In operation, the inflation of the expandable member causes the sheath to expand for release of the coating and/or therapeutic agent through the porous wall or apertures to the tissue of the arterial wall. Preferably, the sheath has a thickness less than 10 mils. However, other thicknesses are possible. Other configurations of suitable sheaths are known in the art. For example, see U.S. Pat. No. 5,370,614 to Amundson, the disclosure of which is incorporated by reference in its entirety.

In another embodiment, the sheath has at least one longitudinal line of weakness allowing the sheath to rupture upon inflation of the balloon and the release of the coating and/or therapeutic agent onto the tissue of the arterial wall of the vessel. Preferably, the sheath material is an elastomeric material which will spring back to a smaller dimension when split to expose more of the body lumen to the coating. The line of weakness can be provided by various techniques known in the art. However, one non-limiting example includes perforating the sheath material. In operation, the sheath is stretched and placed over the coated balloon while in the deflated state. When the coated balloon is inflated, the sheath is expanded beyond its elastic limit at the line of weakness and bursts to expose and therefore release the coating and/or therapeutic agent to the tissue of the arterial wall or vessel lumen. For example, see U.S. Pat. No. 5,370,614 to Amundson, the entire disclosure of which is incorporated by reference.

In accordance with another aspect of the disclosed subject matter, a therapeutic agent is disposed on at least the expandable member or the tissue engaging member, or both. The therapeutic agent can be for the treatment of a disease. Examples of suitable therapeutic agents include anti-proliferative, anti-inflammatory, antineoplastic, antiplatelet, anti-coagulant, anti-fibrin, antithrombotic, antimitotic, antibiotic, antiallergic and antioxidant compounds. Such therapeutic agents can be, again without limitation, a synthetic inorganic or organic compound, a protein, a peptide, a polysaccharides and other sugars, a lipid, DNA and RNA nucleic acid sequences, an antisense oligonucleotide, an antibodies, a receptor ligands, an enzyme, an adhesion peptide, a blood clot agent including streptokinase and tissue plasminogen activator, an antigen, a hormone, a growth factor, a ribozyme, and a retroviral vector.

Preferably, however, the therapeutic agents include a cytostatic drug. The term “cytostatic” as used herein means a drug that mitigates cell proliferation but allows cell migration. These cytostatic drugs, include for the purpose of illustration and without limitation, macrolide antibiotics, rapamycin, everolimus, zotaroliumus, biolimus, temsirolimus, deforolimus, novolimus, myolimus, structural derivatives and functional analogues of rapamycin, structural derivatives and functional analogues of everolimus, structural derivatives and functional analogues of zotarolimus and any marcrolide immunosuppressive drugs. The term “cytotoxic” as used herein means a drug used to inhibit cell growth, such as chemotherapeutic drugs. Some non-limiting examples of cytotoxic drugs include vincristine, actinomycin, cisplatin, taxanes, paclitaxel, and protaxel. Other preferred drugs include dexamethasone, statins, sirolimus, and tacrolimus.

In addition to the therapeutic agent, any of a variety of fluid compositions can be applied to the expandable member or the tissue engaging member, or both. The fluid can include compounds or additives, such as polymers, binding agents, plasticizers, solvents, surfactants, additives, chelators, fillers, excipients, and the like, or combinations thereof. Suitable excipients, binding agents and other components include those described in detail in U.S. patent application Ser. No. 12/636,079, which is hereby incorporated by reference in its entirety. Preferred excipients include poly(ethylene glycol) (PEG), polyvinylpyrrolidone (PVP), polyoxyethylene sorbitan monooleate (tweens), poloxamer triblock copolymers of poly(ethylene oxide)-poly(propylene oxide)-poly(ethylene oxide) (Pluronics), carboxymethyl cellulose (CMC), and PEG phospholipids such as 1,2-distearolyl-sn-glycero-3-phosphoethanolamine-N-(methoxy(polyethylene glycol)-2000) (PEG-PE). Preferred plasticizers include PEG, propylene glycol, N-methylpyrrolidone (NMP), glycerin, and tweens. Examples of possible compounds include zotarolimus, PVP and glycerol. In one embodiment the therapeutic agent can be provided in liquid form or dissolved in a suitable solvent. In another embodiment, the therapeutic agent is provided as a particulate and mixed in a suitable carrier for application as a fluid.

The fluid compositions, such as the therapeutic agents, can be applied to the expandable member or the tissue engaging member using a variety of know techniques, such as spraying (air-atomization, ultrasonic, electrostatic, piezoelectric, etc.), spray drying, pneumatic spray, spray with patterning, electrospinning, direct fluid application, dip-coating, spin-coating, pipette coating, syringe coating, vapor deposition, roll coating, micro-droplet coating, ultrasonic atomization, or other means as known to those skilled in the art. The coating can be applied over at least a length or the entirety of the expandable member. By way of example, and not limitation, certain coating processes that can be used with the instant disclosed subject matter are described in U.S. Pat. No. 6,669,980 to Hansen; U.S. Pat. No. 7,241,344 to Worsham; U.S. Publication No. 2004/0234748 to Stenzel; and U.S. Patent Application Ser. No. 61/345,575, the entire disclosures of which are hereby incorporated by reference. In accordance with one embodiment of the disclosed subject matter, the coating can be applied to either a folded or inflated balloon. Furthermore, the coating can be directly applied into the folds of the folded balloons. The coating characteristics are affected by process variables. For example, for dip-coating process, coating quality and thickness can vary as an effect of variables such as number, rate, and depth of dips along with drying time and temperature.

In accordance with another aspect of the disclosed subject matter, the expandable member or tissue engaging member can include microcapsules on its outer surface. In this regard, the microcapsules are configured to encompass the coating and/or therapeutic agent. Upon inflation of the expandable member the microcapsules located on the surface of the expandable member contact the tissue of the arterial wall. Alternatively, the microcapsules can be formed in the wall of the expandable member surface or on the tissue engaging member. The coating and/or therapeutic agent can be released from the microcapsules by fracturing of the microcapsules and/or diffusion from the microcapsule into the arterial wall. The microcapsules can be fabricated in accordance with the methods disclosed in U.S. Pat. No. 5,1023,402 to Dror or U.S. Pat. No. 6,129,705 to Grantz and the patents referenced therein, each of which is incorporated herein by reference in its entirety.

After positioning the portion of the medical device including the expandable member and tissue engaging member to a select location within the vasculature, the tissue engaging member is deployed. For example, and with reference to the embodiment of FIG. 6B, the tissue engaging member can be deployed by displacement of the sheath relative the tissue engaging member. For example, FIG. 6b shows the tissue engaging member 40 deployed after the cylindrical sheath 50 is moved longitudinally in the proximal direction along the tubular member 12. Since the tissue engaging member of this embodiment is a self-expanding structure, it will expand upon displacement of the sheath, as shown in FIG. 6b. In one embodiment, the tissue engaging member will expand until it contacts the vessel wall. Alternatively, the tissue engaging member can only partially expand such that it does not contact the vessel wall, and thus tissue engagement will occur upon expansion of the expandable member.

After deploying the tissue engaging member, the expandable member is expanded to engage the therapeutic agent with a vessel wall. Any techniques known in the art for expanded the expandable member can be used. For example, if the expandable member is a balloon, an inflation lumen located within the tubular member can supply an inflation medium under positive pressure to the expandable member, thus causing the expandable member to inflate. The expandable member can be inflated to a diameter about equal to the diameter of a reference vessel or up to about 30% larger that the diameter of the reference vessel. The expandable member can be inflated for about 5 minutes or less, depending on the treatment performed and the location of the lumen in the body.

Inflating the expandable member will cause the expandable member to contact the vessel wall and the therapeutic agent will be rapidly released. Furthermore, inflating the expandable member can engage the tissue engaging member with the vessel wall. Even if a self-expanding tissue engaging member is used, the inflation can urge the tissue engaging member further into the tissue of the vessel wall to assist in increasing the therapeutic agent transfer. The tissue engaging member preferably is designed to transmit force evenly about the circumference of the vessel wall, thus causing controlled injury to the vasculature, which increases the efficiency the transfer of therapeutic agent to the body lumen. Thus, the method described herein can provide a controlled angioplasty treatment to the vessel wall and drug delivery and transfer to the vessel wall in one step. However, if desired a predilation step can be performed. Furthermore, the expandable member can undergo multiple inflations, and/or the device can be rotated during or between inflations.

After the expandable member has been inflated for a sufficient time to widen the obstructed vessel wall and/or to transfer the therapeutic drug to the vessel wall, the expandable member is deflated. A variety of techniques known in the art for deflating the expandable member can be used. For example, an inflation lumen located within the tubular member can withdraw the inflation medium, i.e. provide negative pressure, from the expandable member thus causing the expandable member to deflate. A number of known devices and techniques can be used for withdrawing desired amounts of inflation medium. For example, a deflation device such as a syringe pump, having a gas-tight syringe can be attached to the inflation lumen of the expandable member. The deflation device allows for automated, repeatable, and controlled amount of fluid withdrawn by volume from the expandable member. This is advantageous since it reduces or eliminates the variability inherent in a human operator controlled method or apparatus. Alternative devices include an indeflator or vacuum box to draw a vacuum on the expandable member. The indeflator or vacuum box is placed in fluid communication with the inflation lumen of the expandable member to remove the fluid located in the expandable member.

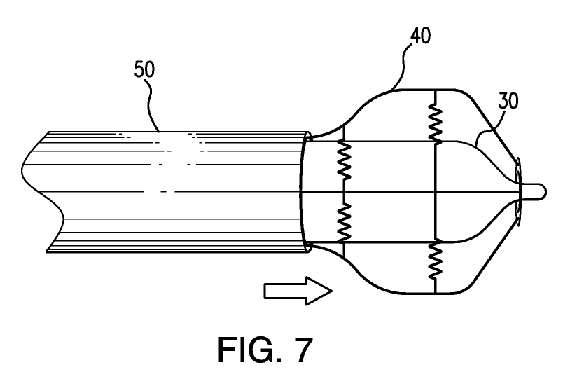

After deflating the expandable member, the medical device is withdrawn from the vasculature. Preferably, the sheath can be replaced so that it is disposed over the tissue engaging member before withdrawing the tubular member from the vasculature. Alternatively, the device can be withdrawn without replacing the sheath. In accordance with one aspect of the disclosed subject matter, if the tissue engaging member is self-expandable, the medical device can include hooks or any other suitable configuration to collapse tissue engaging member after deflation but before withdrawing the medical device from the vasculature. For example, the sheath can be used to assist in the collapse of the tissue engaging member, after deflation, as shown in FIG. 7 for the purpose of illustration and not limitation.

In accordance with another aspect of the disclosed subject matter, an alternative medical device and method of delivering a therapeutic agent is provided. The medical device includes a tubular member having a proximal end and distal end defining a longitudinal axis therebetween, an expandable member proximate the distal end of the tubular member, a tissue engaging member proximate the expandable member and having at least one wire disposed within at least one lumen extending along the longitudinal axis of the tubular member, and a therapeutic agent disposed on at least the expandable member or the tissue engaging member. The method includes delivering at least a portion of a medical device within a vasculature, deploying the at least one wire of the tissue engaging member from within the at least one lumen at a select location, inflating the expandable member to engage the therapeutic agent with a vessel wall, deflating the expandable member, and withdrawing the medical device from the vasculature. The method can include any of the features described herein above.

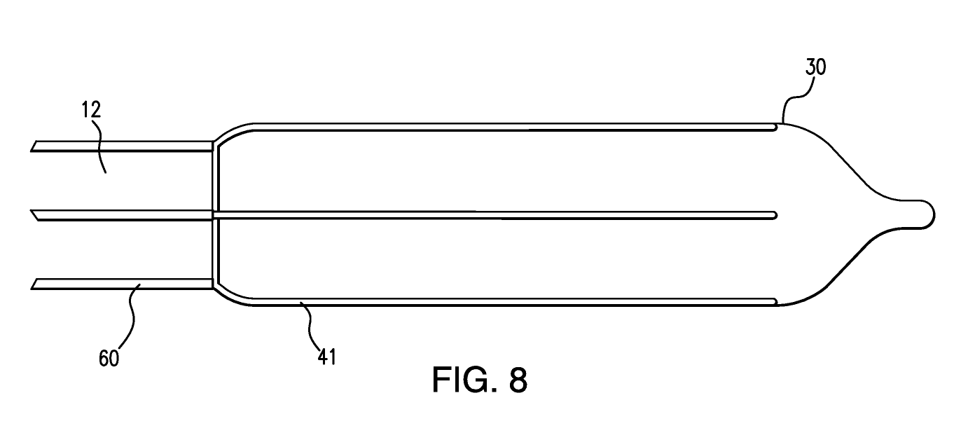

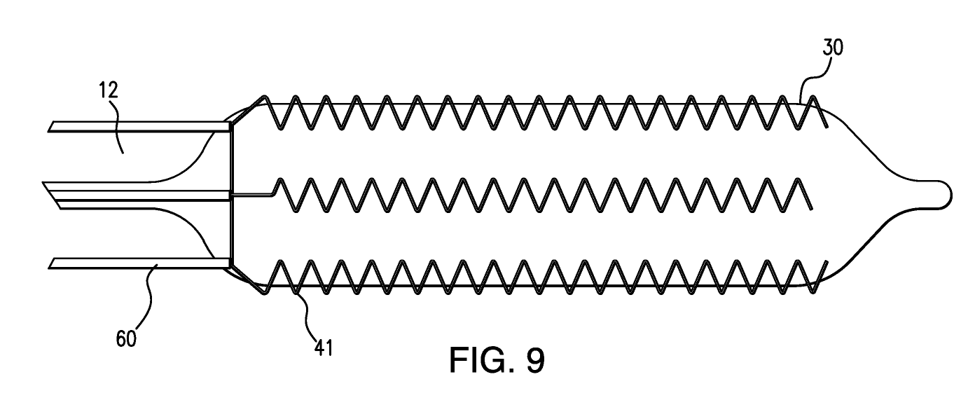

For purpose of explanation and illustration, and not limitation, an exemplary embodiment of the expandable member having a tissue engaging member having at least one wire extending from lumens is shown schematically in FIGS. 8 and 9. Generally, the medical device includes a tubular member and an expandable member of similar construction as previously described. Additionally, one or more lumens 60 are provided along at least a length of the tubular member 12. Contained within each lumen is a wire or element 41 of the tissue engaging member 40. The wire or element 41 is movable between a retracted position and an extended position. During initial positioning of the device, the wire or elements are disposed within the lumen. When at the desired site, the wire or elements are extended so as to be disposed proximate or adjacent to the expandable member. For example, and as shown in FIG. 8, the at least one wire 41 of the tissue engaging member 40 can extend from within lumens 60 extending along the longitudinal axis of the tubular member. The lumens 60 can have any suitable configuration. For example, the lumens can be located on the outside of the tubular member, as shown in FIGS. 8 and 9, and can be attached to the tubular member by any means described above or otherwise known in the art. Alternatively, the lumens 60 can be formed or located within the tubular member.

A variety of actuators can be used to move the at least one wire of the tissue engaging member between the retracted position and the extended position. For example, the wires or elements of the tissue engaging member can be moved or otherwise actuated by a push-pull handle assembly. Alternatively or additionally, an actuator wire can extend the length of the tubular member and can connect to the tissue engaging member by a collar or the like.

It is noted that the medical device of this arrangement can include a number of the features described herein above. For example, at least one tissue engaging member can be a plurality of straight wires 41 as shown in FIG. 8, for the purpose of illustration and not limitation. Alternatively, the wires can have a memory, such as nitinol wires, such that the wires emerge as non-linear wires 41 from the lumens 60, as shown in FIG. 9. Furthermore, the tissue engaging member can have a circumferential wire interconnecting each of the wires located at the distal end of the wires to hold the wires in place during extension from the lumens and during expansion of the expandable member. In this regard, the actuator wire can extend through the expandable member and be jointed to the distal end of the tissue engaging member. After deployment and inflation, the tissue engaging member can be retracted within the lumens before withdrawing the medical device from the vasculature.

While the disclosed subject matter is described above in connection with the delivery of a therapeutic agent to a vasculature, the devices and methods described herein can be used without a therapeutic agent. For example, the methods and devices described herein can be used in angioplasty procedures without drug delivery. Use of an expandable member having a tissue engaging member as described herein provides a controlled angioplasty procedure and improved vascular response to reduce the occurrence of negative side effects (dissections, focal vessel damage, stenosis, and restenosis). For example, the wires anchoring the balloon in place during the angioplasty procedure can distribute the force of the balloon in a controlled manner, thus reducing trauma and increasing uniformity of injury to the vasculature.

Thus, the disclosed subject matter also includes a medical device and a method of treating a vasculature. The method includes delivering at least a portion of a medical device within a vasculature. The medical device includes a tubular member having a proximal end and distal end defining a longitudinal axis therebetween, an expandable member proximate the distal end of the tubular member, a tissue engaging member proximate the expandable member, and a sheath disposed over the tissue engaging member. The method further includes deploying the tissue engaging member at a select location by displacement of the sheath relative the tissue engaging member, inflating the expandable member to engage the expandable member with a vessel wall, deflating the expandable member, and withdrawing the medical device from the vasculature. The medical device and method can include any of the features described above.

Further in accordance with the disclosed subject matter, an alternative medical device and method of treating a vasculature is provided. The method includes delivering at least a portion of a medical device within a vasculature. The medical device includes a tubular member having a proximal end and distal end defining a longitudinal axis therebetween, an expandable member proximate the distal end of the tubular member, and a tissue engaging member proximate the expandable member and having at least one wire disposed within at least one lumen extending along the longitudinal axis of the tubular member. The method further includes deploying the at least one wire of the tissue engaging member from within the at least one lumen at a select location, inflating the expandable member to engage the expandable member with a vessel wall, deflating the expandable member, and withdrawing the medical device from the vasculature. The medical device and method can include any of the features described above.

While the disclosed subject matter is described herein in terms of certain preferred embodiments, those skilled in the art will recognize that various modifications and improvements can be made to the disclosed subject matter without departing from the scope thereof. Moreover, although individual features of one embodiment of the disclosed subject matter can be discussed herein or shown in the drawings of the one embodiment and not in other embodiments, it should be apparent that individual features of one embodiment can be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A method of delivering a therapeutic agent comprising:
delivering at least a portion of a medical device within a vasculature, the medical device including:
a tubular member having a proximal end and distal end defining a longitudinal axis therebetween,
an expandable member proximate the distal end of the tubular member and including an exterior surface,
a tissue engaging member extending from the tubular member and disposed over the exterior surface of the expandable member, the tissue engaging member including at leasₜ one wire element,
a sheath movable between a retracted position and an extended position, the sheath disposed over the tissue engaging member when in the extended position, the tissue engaging member configured for expansion in a radial direction when the sheath is in the retracted position, and
a therapeutic agent disposed on at least one of the expandable member or the tissue engaging member;
deploying the tissue engaging member at a select location by displacement of the sheath;
inflating the expandable member to engage the therapeutic agent with a vessel wall;
deflating the expandable member; and
withdrawing the medical device with the tissue engaging member from the vasculature.

2. The method of claim 1, wherein the tissue engaging member is a self-expanding structure.

3. The method of claim 1, wherein a proximal end of the tissue engaging member is fixedly attached to the tubular member proximate a proximal end of the expanded member.

4. The method of claim 3, wherein a distal end of the tissue engaging member is fixedly attached to the tubular member proximate a distal end of the expanded member.

5. The method of claim 1, wherein the tissue engaging member is coupled to the expandable member such that the tissue engaging member collapses upon deflation of the expandable member.

6. The method of claim 1, wherein the therapeutic agent includes an excipient, plasticizer, or surfactant, or combinations thereof.

7. The method of claim 1, wherein inflating the expandable member further engages the tissue engaging member with the vessel wall.

8. The method of claim 1, further comprising replacing the sheath so that it is disposed over the tissue engaging member before withdrawing the tubular member from the vasculature.

9. The method of claim 1, wherein the expandable member is inflated to a diameter about equal to the diameter of a reference vessel or up to about 30% larger that the diameter of the reference vessel.

10. The method of claim 1, wherein the expandable member is inflated for about 5 minutes or less.

11. The method of claim 1, wherein the tissue engaging member has a tissue engaging member to artery ratio in an expanded state is between about 1% and about 50%.

12. The method of claim 1, wherein the tissue engaging member has a tissue engaging member to artery ratio in an expanded state is between about 2.5% and about 25%.

13. The method of claim 1, wherein the at least one wire element comprises at least one wire having a diameter or width between about 0.05 microns to about 250 microns.

14. A medical device comprising:
a tubular member having a proximal end and distal end defining a longitudinal axis therebetween;
an expandable member proximate the distal end of the tubular member and including an exterior surface;
a tissue engaging member extending from the tubular member and disposed over the exterior surface of the expandable member, the tissue engaging member including at least one wire element;
a retractable sheath movable between a retracted position and an extended position, the retractable sheath disposed over the tissue engaging member when in the extended position; and
a therapeutic agent disposed on at least one of the expandable member or the tissue engaging member;
wherein the tissue engaging member is configured for expansion in a radial direction when the retractable sheath is in the retracted position.

15. The medical device of claim 14, wherein the tissue engaging member is a self-expanding structure.

16. The medical device of claim 15, wherein the tissue engaging member includes a surface feature configured to operatively engage the retractable sheath for conversion between expanded and unexpanded configurations upon displacement of the retractable sheath.

17. The medical device of claim 14, wherein the tissue engaging member is coupled to the expandable member such that the tissue engaging member collapses upon deflation of the expandable member.

18. The medical device of claim 14, wherein the tissue engaging member is configured with a plurality of raised surface features.

19. The medical device of claim 14, wherein the at least one wire includes a plurality of straight wires.

20. The medical device of claim 14, wherein the at least one wire includes a plurality of non-linear wires.

21. The medical device of claim 14, wherein the at least one wire includes a plurality of wires that interconnect.

22. The medical device of claim 14, wherein the therapeutic agent includes an excipient, plasticizer, or surfactant, or combinations thereof.

23. The medical device of claim 14, wherein a proximal end of the tissue engaging member is fixedly attached to the tubular member proximate a proximal end of the expanded member.

24. The medical device of claim 14, wherein a distal end of the tissue engaging member is fixedly attached to the tubular member proximate a distal end of the expanded member.

25. The medical device of claim 14, wherein the tissue engaging member has a tissue engaging member to artery ratio in an expanded state of between about 1% and about 50%.

26. The medical device of claim 14, wherein the tissue engaging member has a tissue engaging member to artery ratio in an expanded state of between about 2.5% and about 25%.

27. The medical device of claim 14, wherein the at least one wire has a diameter or width between about 0.05 microns to about 250 microns.

28. A method of delivering a therapeutic agent comprising:
delivering at least a portion of a medical device within a vasculature, the medical device including:
a tubular member having a proximal end and distal end defining a longitudinal axis therebetween,
an expandable member proximate the distal end of the tubular member and including an exterior surface,
a tissue engaging member proximate the expandable member and movable between a retracted position and an extended position, the tissue engaging member having at least one wire disposed within at least one lumen extending along a length of the tubular member when in the retracted position, the tissue engaging member disposed over the exterior surface of the expandable member and configured for expansion in a radial direction when in the extended position, and
a therapeutic agent disposed on at least one of the expandable member or the tissue engaging member;
deploying the at least one wire of the tissue engaging member from the retracted position to the extended position at a select location within the vasculature;
inflating the expandable member to engage the therapeutic agent with a vessel wall within the vasculature;
deflating the expandable member; and
withdrawing the medical device with the tissue engaging member from the vasculature.

29. The method of claim 28, wherein the at least wire of the tissue engaging member is extended extend the entire length of the expandable member.

30. A medical device comprising:
a tubular member having a proximal end and distal end defining a longitudinal axis therebetween;
an expandable member proximate the distal end of the tubular and including an exterior surface;
at least one lumen extending along a length of the tubular member;
a tissue engaging member movable between a retracted position and an extended position, the tissue engaging member having at least one wire element slidingly disposed within the at least one lumen when in the retracted position, the tissue engaging member disposed over the exterior surface of the expandable member when in the extended position;
a therapeutic agent disposed on at least one of the expandable member or the tissue engaging member; and
wherein the tissue engaging member is configured for expansion in a radial direction when in the extended position.

31. A method of treating a vasculature comprising:
delivering at least a portion of a medical device within a vasculature, the medical device including:
a tubular member having a proximal end and distal end defining a longitudinal axis therebetween,
an expandable member proximate the distal end of the tubular member and including an exterior surface,
a tissue engaging member extending from the tubular member and disposed over the exterior surface of the expandable member, the tissue engaging member including at least one wire element, and
a sheath disposed movable between a retracted position and an extended position, the sheath disposed over the tissue engaging member when in the extended position, the tissue engaging member configured for expansion in a radial direction when the sheath is in the retracted position;
deploying the tissue engaging member at a select location to the extended position by displacement of the sheath;
inflating the expandable member to engage the expandable member with a vessel wall;
deflating the expandable member; and
withdrawing the medical device with the tissue engaging member from the vasculature.

32. A medical device comprising:
a tubular member having a proximal end and distal end defining a longitudinal axis therebetween;
an expandable member proximate the distal end of the tubular member and including an exterior surface;
a tissue engaging member extending from the tubular member and disposed over the exterior surface of the expandable member, the tissue engaging member including at least one wire element; and
a retractable sheath movable between a retracted position and an extended position, the retractable sheath disposed over the tissue engaging member when in the extended position;
wherein the tissue engaging member is configured for expansion in a radial direction when the retractable sheath is in the retracted position.

33. A method of treating a vasculature comprising:
delivering at least a portion of a medical device within a vasculature, the medical device including:
a tubular member having a proximal end and distal end defining a longitudinal axis therebetween,
an expandable member proximate the distal end of the tubular member and including an exterior surface,
a tissue engaging member proximate the expandable member movable between a retracted position and an extended position, the tissue engaging member having at least one wire disposed within at least one lumen extending along a length of the tubular member when in the retracted position, the tissue engaging member disposed over the exterior surface of the expandable member and configured for expansion in a radial direction when in the extended position;
deploying the at least one wire of the tissue engaging member from the retracted position to the extended position at a select location within the vasculature;
inflating the expandable member to engage the expandable member with a vessel wall within the vasculature;
deflating the expandable member; and
withdrawing the medical device with the tissue engaging member from the vasculature.

34. A medical device comprising:
a tubular member having a proximal end and distal end defining a longitudinal axis therebetween;
an expandable member proximate the distal end of the tubular member and including an exterior surface;
at least one lumen extending along a length of the tubular member; and
a tissue engaging member movable between a retracted position and an extended position, the tissue engaging member having at least one wire element slidingly disposed within the at least one lumen when in the retracted position, the tissue engaging member disposed over the exterior surface of the expandable member when in the extended position;
wherein the tissue engaging member is configured for expansion in a radial direction when in the extended position.
